(19) United States
(12) Patent Application Publication
(10) Pub. No.: US 2025/0098986 A1
(43) Pub. Date: Mar. 27, 2025

(54) SYSTEMS AND METHODS FOR NON-CONTACT RESPIRATORY MONITORING

(71) Applicant: Covidien LP, Mansfield, MA (US)

(72) Inventors: Andre Antunes, Edinburgh (GB); Dean Montgomery, Edinburgh (GB); Paul S. Addison, Edinburgh (GB)

(21) Appl. No.: 18/885,869

(22) Filed: Sep. 16, 2024

Related U.S. Application Data
(60) Provisional application No. 63/539,026, filed on Sep. 18, 2023.

(51) Int. Cl. A61B 5/113; A61B 5/00; A61B 5/11

(52) U.S. Cl. CPC A61B 5/113; A61B 5/1128; A61B 5/7267; A61B 5/742

(57) ABSTRACT

A method and system for performing a depth data processing procedure for obtaining physiological information from depth data. The depth processing procedure comprises obtaining depth data representing depth across a field of view, and deriving at least one signal from said depth data. The method further comprises: obtaining further information from at least one of: the depth data and the at least one signal derived from the depth data; using at least said further information to determine an absence of respiratory motion in the field of view; and setting a flag for the depth data processing procedure or a further procedure based on said determination of the absence of respiratory motion in the field of view.

### CROSS-REFERENCE TO RELATED APPLICATION

The present application claims the benefit of U.S. Provisional Patent Application Ser. No. 63/539,026, filed on Sep. 18, 2023, the entire content of which is incorporated herein by reference.

### FIELD

The present disclosure relates to depth data processing procedures for obtaining physiological information using non-contact respiratory monitoring system, and more particularly, the present disclosure relates to a method and apparatus for setting a flag for a depth data processing procedure based on a determination of an absence of respiratory motion.

### BACKGROUND

A common vital sign measured in a clinical setting is respiratory rate (RR). A significant change in RR is often an early indication of a major complication such as respiratory tract infections, respiratory depression associated with opioid consumption, anaesthesia and/or sedation, as well as respiratory failure.

Video-based monitoring is a field of patient monitoring that uses a remote video camera to detect physical attributes of the patient. This type of monitoring may also be called “non-contact” monitoring in reference to the remote video sensor, which does not contact the patient. A system based on a depth-sensing camera for the continuous non-contact ‘touchless’ monitoring of Respiratory Rate (RR) has been developed. It is known to use depth sensing devices to determine a number of physiological and contextual parameters for patients including respiratory rate, tidal volume, minute volume, effort to breathe, activity, presence in bed. The determination of respiratory rates from depth information may be performed, for example, by identifying breaths in the respiratory volume waveform derived by integrating depth information over a spatial region where respiration occurs, or by the evaluation of the power spectrum of a respiratory signal.

Known depth sensing based respiratory rate algorithms may use respiratory and/or other physiological signals obtained using depth data to generate a value for a respiratory rate or other physiological parameters. The waveforms obtained from the depth data may contain a significant amount of coherent noise. Known algorithms applied to such signals may mistake the noise for respiratory motion in certain circumstances and output a respiratory rate or other parameter even if no respiratory motion is present in the scene. There is a need to avoid display of respiratory rate or other physiological parameters in the absence of actual respiratory motion.

### SUMMARY

According to a first aspect, there is provided a computer-implemented method comprising: performing a depth data processing procedure for obtaining physiological information from depth data, wherein the depth processing procedure comprises obtaining depth data representing depth across a field of view, and deriving at least one signal from said depth data; wherein the method further comprises: obtaining further information from at least one of: the depth data and the at least one signal derived from the depth data; using at least said further information to determine an absence of respiratory motion in the field of view; and setting a flag for the depth data processing procedure or a further procedure based on said determination of the absence of respiratory motion in the field of view.

The depth data may represent depth information. The depth data may comprise data sensed or otherwise obtained by a depth sensing device. The depth data and/or depth data may be obtained as a function of position across the field of view of a depth sensing device. The depth data may be obtained from a region of interest in the field of view. The depth data may be obtained from an identified region in the field of view, for example, a mask region. The processor may be configured to identify a region of interest. The physiological information and/or at least one signal may be obtained by processing depth data from the region of interest. The depth data may represent a distance between a subject and/or other object in the field of view and the depth sensor. Performing the procedure may comprise receiving data, for example, the further information, output by the procedure. The depth data processing procedure may comprise obtaining physiological information form the at least one signal. The further information may comprise further data obtained or generated during the operation of the depth data processing procedure.

The depth data processing procedure may be configured to extract a signal related to respiration from the depth data.

The signal related to respiration may comprise, for example, a signal related to respiratory rate, pulse rate, tidal volume, minute volume, effort to breathe, oxygen saturation or any breathing parameter.

The at least one signal derived from the depth data may comprise a first, time dependent signal and a second, frequency dependent signal, and wherein the further information comprises information obtained from the time dependent signal and information obtained from the frequency dependent signal, and optionally information from the depth data.

The further information may comprise further data obtained or generated by the procedure. The further information may comprise parameters relating to performance of the procedure.

Deriving the at least one signal may comprise deriving a first time dependent signal from the depth data wherein the at least one signal is represented by a waveform or other graphical representation at least one signal is represented by a waveform or other graphical representation and wherein the further information comprises one or more properties of a waveform.

The at least one signal may comprise a further signal obtained from a power spectral analysis and/or frequency analysis of the time dependent signal and the further information comprises one or more properties of the further signal. The at least one signal may comprise a power spectrum

The at least one signal may comprise a respiratory signal and a power spectrum signal obtained from the respiratory signal, wherein the further information comprises one or more properties of at least one of: the depth data, the respiratory signal, the power spectrum.

The further information may comprise information derived from a first signal obtained from the depth data over a first time window and a second signal obtained from the depth data over a second time window, wherein the method further comprises selecting the first and/or second time windows.

The method may comprise determining whether the at least one signal is representative of no respiratory motion within a pre-determined time window. The further information may comprise values for one or more pre-determined features associated with the depth data, the time varying signal and the transformed signal.

The flag may be set in response to determining absence of the respiratory motion. The at least one signal may comprise a power or spectral density, for example, as a function of frequency and/or a power or energy per unit time signal

The physiological information may comprise a physiological signal or physiological parameter signal, for example, a signal representing a respiratory rate, pulse rate, tidal volume, minute volume, effort to breathe, oxygen saturation, a breathing parameter or other vital sign, posture information, sleep apnea information.

The procedure may further comprise obtaining contextual or other subject information associated with a subject. Contextual information include, for example, presence information or activity information. Subject information may comprise identify information.

The further information may represent at least one or more properties, characteristics or features of the depth data and/or the at least one signal.

The further information for the at least one signal may comprises at least one of a), b), c), d), e), f), g):
a) an area under at least part of, optionally all, of the signal;
b) an average value for at least part of, optionally all of the signal
c) one or more properties associated with or derived from one or more peaks and/or troughs of the signal;
d) a property associated with a zero crossing of the signal;
e) a measure of bias or noise in the signal;
f) a count or density of one or more identified features in the signal;
g) at least one further mathematical property of the signal.

The method may comprise performing one or more feature value extraction processes on output data from and/or intermediate data obtained during the procedure to determine the physiological information.

The at least one property of one or more peaks and/or troughs may comprise: an area of one or more peaks; a height of one or more peaks; a spread of one or more peaks; a number and/or density of peaks in the signal; a ratio between a property of one peak and the same property of another peak. The area may be an integral or summed quantity of at least part of the signal. The height may be an intensity of magnitude. The spread may correspond to a time or frequency range. The number and/or density of peaks may be determined relative to a threshold value. The at least one property may comprise a relative area, size or spread of a first peak to a second peak.

The at least one signal may comprise a power spectrum and the further information may comprise at least of one of: a measure of total power in the spectrum, a power of a first main peak and/or a second main peak, a number of peaks above a threshold, a percentage of power in a first main peak and/or second main peak, a relative power or ratio of power between one or more peaks, a frequency of one or more peaks of the spectrum

The at least one signal may comprise a time varying signal represented as a waveform and/or other graphical representation and the further information may comprise at least one of: a measure of the amplitude and/or size of the signal; a number and/or density of zero crossings of the signal; a property of one or more peaks and/or troughs of the waveform; a measure or trend of noise or bias in the signal.

The procedure may comprise determining one or more portions of the depth data corresponding to coherent changes in changes or otherwise obtaining a mask for a visual overlay, wherein the further information comprises at least one property of the one or more portions and/or the determined mask, optionally wherein the at least one property comprises: a size, a shape, a fill ratio.

The determination of the absence may comprise applying a classification procedure to the obtained further information to classify the further information as representative or at least indicative of the absence of respiratory motion. The classification procedure may comprise a deterministic and/or machine-learning derived classifier

The classifier may comprise a machine learning derived model and/or classifier, for example, decision tree, kNN, AdaBoost, Random Forest, Neural Network, SVM.

The determination of the absence may comprise applying a threshold based algorithm comprising comparing the further information to one or more thresholds and wherein the method further comprises determining threshold values for the threshold based algorithm using training data.

Processing the further information may comprise comparing one or more properties, characteristics or features of the at least one signal and/or depth data to a threshold value wherein the result of the comparison is at least indicative of the absence of respiratory motion in the field of view.

The depth data processing procedure may comprise displaying said physiological information wherein the determination and/or the display of the physiological information is in dependence on the flag.

The depth data processing procedure may be configured to not display one or more parameters in dependence on the flag.

The depth data processing procedure may comprise a procedure for the determination of a respiratory rate, wherein the method comprises displaying said respiratory rate in dependence on said flag. The depth data processing procedure may comprise a procedure for determination of a mask or other visual overlay representing respiration and wherein the method comprises displaying the mask or other visual overlay in dependence on the flag.

The method may comprise performing at least one action in dependence on said flag. The at least one action may comprise triggering an alarm signal. The alarm signal may be a visual or audible alarm.

The further information may relate to one or more signal derived from said depth data as part of the procedure and wherein the method comprises pre-processing the one or more signal and obtaining said further information from the pre-processed at least one signal, wherein pre-processing comprises filtering and/or selecting and/or cleaning of said at least one signal.

The method may comprise repeating said determination of the absence and storing results of said determination in a memory storage buffer wherein the setting of said flag is based on an evaluation of stored results in the buffer.

The method may comprise obtaining the depth data using at least one of: a depth sensing camera, a stereo camera, a camera cluster, a camera array, a motion sensor.

According to a second aspect, there is provided a depth sensing device configured to obtain depth data representing depth across a field of view; a processing resource configured to: perform a depth data processing procedure for obtaining physiological information from the depth data wherein the depth data processing procedure comprises deriving at least one signal from said depth data; wherein the processing resource is further configured to: obtain further information from at least one of: the depth data and the at least one signal derived from the depth data; use said further information to determine an absence of respiratory motion in the field of view; and set a flag for the depth data processing procedure and/or performing at least one further action based on said determination of the absence of respiratory motion in the field of view.

The depth sensing device may comprise at least one of: a depth sensing camera, a stereo camera, a camera cluster, a camera array, a motion sensor.

The apparatus may further comprise a display and the display of at least the physiological information may be in dependence on the value of the flag.

The processing resource may comprise at least one first processor configured to perform the procedure and at least one further processor configured to derive said further information and determine an absence of respiratory motion. The processing resource may be configured extract said further information as part of the depth data processing procedure.

According to a third aspect, there is provided a non-transitory machine-readable medium having instructions recorded thereon for execution by a processor to: perform a depth data processing procedure for obtaining physiological information from depth data, wherein the depth processing procedure comprises obtaining depth data representing depth across a field of view, and deriving at least one signal from said depth data; obtain further information from at least one of: the depth data and the at least one signal derived from the depth data; use said further information to determine an absence of respiratory motion in the field of view; and set a flag for the depth data processing procedure and/or performing at least one further action based on said determination of the absence of respiratory motion in the field of view.

Features in one aspect may be applied as features in any other aspect, in any appropriate combination. For example, method features may be applied as apparatus features or non-transitory machine-readable medium features, and vice versa.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1. is a schematic view of a patient monitoring system for monitoring a subject, the system having a depth sensing camera, in accordance with various embodiments;

FIG. 2 is a block diagram that illustrates a patient monitoring system having a computing device, a server, one or more image capture devices and configured in accordance with various embodiments;

FIG. 3A is a rendered image of a patient monitoring system and support apparatus and FIG. 3B depicts the patient monitoring system and support apparatus adjacent to a hospital bed, in accordance with various embodiments;

FIG. 4 is screenshot of a display of the system, in accordance with various embodiments, depicting an image captured by a camera and an obtained physiological information;

FIG. 5A is a graph showing a respiratory waveform over time and FIG. 5B is a graph of a power spectrum obtained from the waveform of FIG. 5A;

FIG. 6A is a graph showing a further respiratory waveform over time and FIG. 6B is a graph of a further power spectrum obtained from the waveform of FIG. 6A;

FIG. 7 is a flow-chart showing a method of setting a flag for a depth data processing procedure, and

FIG. 8 is a flow-chart of a classification method, in accordance with various embodiments.

### DETAILED DESCRIPTION

Known depth sensing based respiratory rate algorithms may use respiratory and/or other physiological signals obtained using depth data to generate a value for a respiratory rate or other physiological parameters. The waveforms can contain a significant amount of coherent noise that can be easily mistaken for respiratory motion, even if there is no subject present in the scene. In such situations, known respiratory rate (RR) algorithms may erroneously estimate and post an RR value even when there is no respiratory activity taking place in the scene. No respiratory activity may correspond to a patient having ceased breathing or having left the scene.

The present disclosure relates to a system and method for detecting and classifying signals when there is no respiratory motion in the scene in order to set a flag indicating no respiratory motion (referred to as a NRM flag). In response to setting the NRM flag, the processor may take one or more further actions. By setting a flag, the system may prevent the erroneous display, or calculation, of an RR value when no respiratory motion is present in the scene. The present disclosure therefore relates to a system and method that may avoid display of respiratory rates or other physiological parameters in the absence of actual respiratory motion or activity. Alternative approaches to avoiding erroneous display of respiratory rates include using known object or subject identification algorithms to identify a presence of patients in the field of view. These approaches may be undesirable due to, for example, the difficulty of reliably identifying patients in a clinical environment due to obstructions and because an identification of a patient does not correspond to a guarantee of respiratory motion. In addition, such approaches may add additional complexity to the respiratory rate system, for example, they may require additional image capture devices.

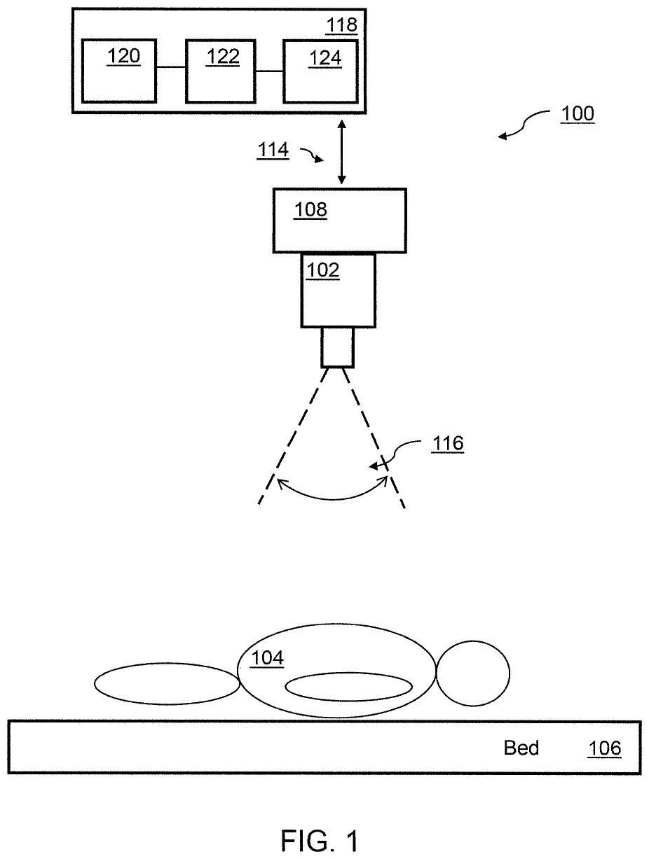

FIG. 1 is a schematic view of a patient monitoring system 100 for monitoring a subject, for example patient 104. The patient monitoring system 100 may be provided in a number of settings. In the present embodiment, the system 100 is described in the context of a clinical environment, for example, a hospital, and is provided adjacent to a hospital bed 106.

The system 100 includes a non-contact depth determining device, in particular, an image-based depth sensing device. In the present embodiment, the image-based depth sensing device is a depth-sensing camera 102. The camera 102 is at a remote position from the patient 104 on the bed 106. The camera 102 is remote from the patient 104, in that it is spaced apart from the patient 104 and does not contact the patient 104. In particular, the camera 102 is provided at an elevated position at a height above the bed and angled to have a field of view of at least part of the bed 106. It will be understood that, while FIG. 1 depicts a single image capture device (camera 102) the system may have more than one image capture or depth sensing devices.

In the embodiment of FIG. 1, the field of view of the camera 102 includes at least an upper portion of the bed 106 such that, in use, a torso or chest region of the patient 104 is visible in the field of view 116, to allow respiratory information to be obtained using obtained depth data.

The camera 102 generates a sequence of images over time. In the described embodiments, the camera 102 is a depth sensing camera, such as a Kinect camera from Microsoft Corp. (Redmond, Wash.) or a RealSense™ depth camera from Intel (Intel, Santa Clara, California). A depth sensing camera can detect a distance between the camera and objects in its field of view. This depth information can be used, as disclosed herein, to determine a region of interest (ROI) to monitor on the patient. Once a ROI is identified, that ROI can be monitored over time, and depth data associated with the region of interest obtained. The obtained depth data is representative of depth information as a function of position across the field of view of the camera.

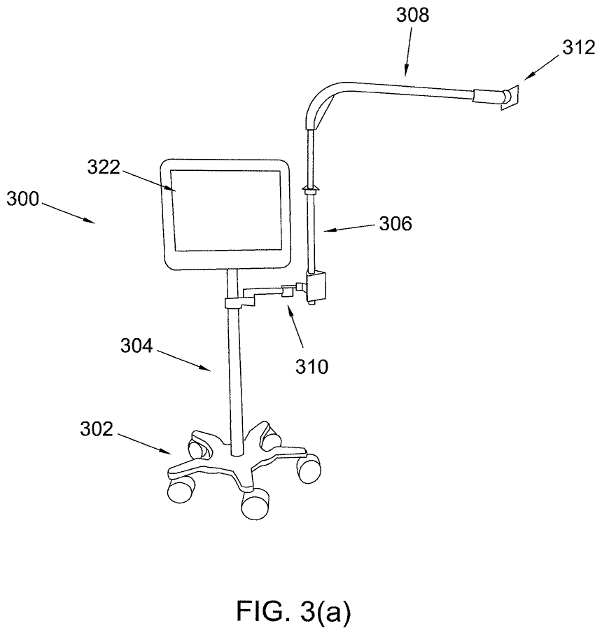

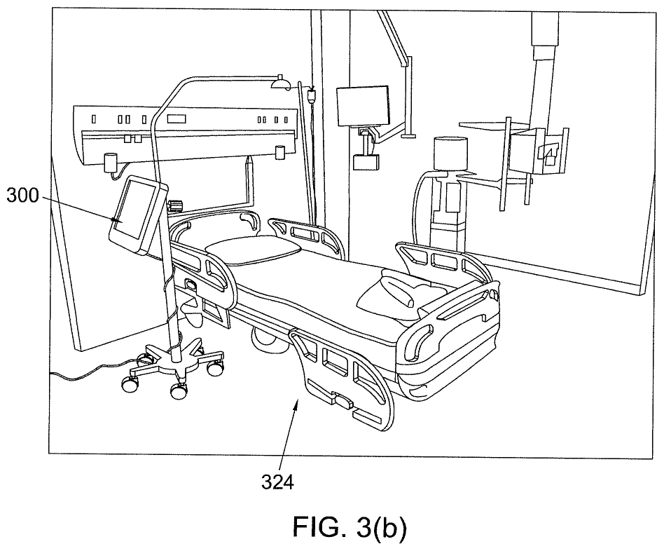

The camera 102 is provided on a support apparatus 108. An example of a support apparatus is described with reference to FIGS. 3A and 3B). The support apparatus 108 supports the camera 102 at a depth sensing position above the patient 102. At the depth sensing position, the camera 102 has a field of view that includes at least part of the bed 106 and at least part of the patient 104 (including the desired region of interest).

In the embodiments described herein, the depth data is processed to extract time-varying depth information for a region of interest of the patient, for example, the depth data of a chest region of the patient is processed to obtain time-varying depth information associated with respiration of the patient. However, it will be understood that in some embodiments, the time-varying depth information may be associated with other physiological functions or processes of the patient. As described below, the depth data may also be processed to determine physiological information about the patient.

In some embodiments, physiological information about the patient is extracted by processing the depth data in accordance with known depth data processing techniques. A review of known depth data processing techniques is provided in “Noncontact Respiratory Monitoring Using Depth Sensing Cameras: A Review of Current Literature”, Addison, A. P., Addison, P. S., Smit, P., Jacquel, D., & Borg, U. R. (2021). Sensors, 21 (4), 1135. The physiological information may include, for example, information related to respiration, breathing or heart rate, for example, respiratory rate, pulse rate, tidal volume, minute volume, effort to breathe, oxygen saturation or any breathing parameter or vital sign. Physiological information may include any parameter or signal associated with the functioning of the body. The physiological information may be obtained from the depth data using known depth data processing techniques. The present disclosure relates to methods and system for setting a flag for a depth data processing procedure.

The image-based depth sensing device may have depth sensor elements that sense light having infra-red wavelengths. The depth sensor elements may sense electromagnetic radiation having wavelengths in the range 1 mm to 700 nm. While an infra-red wavelength depth sensing camera is described, it will be understood that other wavelengths of light or electromagnetic radiation may be used.

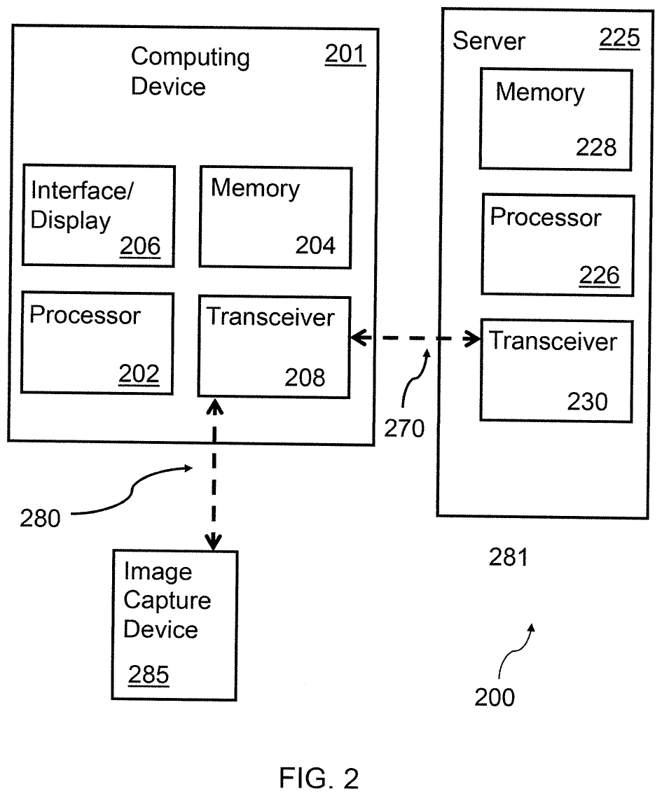

While only a single camera is depicted in FIG. 1 and FIG. 2 it will be understood that, in some embodiments, multiple cameras may be mounted or positioned about the patient.

The field of view of the camera may be defined by a first subtended angle and a second subtended angle. The first and second subtended angles may be in the range, for example, 10 to 100 degrees. In further embodiments, the first and second subtended angles may be in the range 40 to 95 degrees. The first and second subtended angles may be in the range, for example, 10 to 100 degrees. In further embodiments, the first and second subtended angles may be in the range 40 to 95 degrees.

While the camera 102 may be a depth sensing camera, in accordance with various embodiments, any image-based or video-based depth sensing device may be used. For example, a suitable depth sensing device may be a depth sensor that provides depth data for object in the field of view. In some embodiments, the system has an image capture device for capturing images across its field of view together with an associated depth sensor that provides depth data associated with the captured images. The depth information is obtained as a function of position across the field of view of the depth sensing device.

In some embodiments, the depth data can be represented as a depth map or a depth image that includes depth information of the patient from a viewpoint (for example, the position of the image capture device). The depth data may be part of a depth data channel that corresponds to a video feed. The depth data may be provided together with image data that comprises RGB data, such that each pixel of the image has a corresponding value for RGB and depth. The depth data may be representative or indicative of a distance from a viewpoint to a surface in the vehicle. This type of image or map can be obtained by a stereo camera, a camera cluster, camera array, or a motion sensor. When multiple depth images are taken over time in a video stream, the video information includes the movement of the points within the image, as they move toward and away from the camera over time.

The captured images, in particular, the image data corresponding to the captured images and the corresponding depth data are sent to a computing device 118 through a wired or wireless connection 114. The computing device 118 includes a processor 120, a display 122, and hardware memory 124 for storing software and computer instructions. Sequential image frames of the occupant are recorded by the camera 102 and sent to the processor 120 for analysis. The display 122 may be remote from the camera 102, such as a video screen positioned separately from the processor and memory. The processor 120 is configured to perform one or more pre-determined data depth processing procedures or algorithms on depth data from the camera. As described in the following, the processor 120 is further configured processor to set a flag parameter for such procedures in response to detecting the absence of respiratory motion in the field of view.

Other embodiments of the computing device may have different, fewer, or additional components than shown in FIG. 1. In some embodiments, the computing device may be a server. In other embodiments, the computing device of FIG. 1 may be additionally connected to a server (e.g., as shown in FIG. 2 and discussed below). The depth data associated with the images/video can be processed or analysed at the computing device and/or a server to obtain time-varying depth information for the patient.

FIG. 2 is a block diagram illustrating a patient monitoring system 200, having a computing device 201, a server 225 and an image capture device 285 according to embodiments. In various embodiments, fewer, additional and/or different components may be used in a system.

The computing device 201 includes a processor 202 that is coupled to a memory 204. The processor 202 can store and recall data and applications in the memory 204, including applications that process information and send commands/signals according to any of the methods disclosed herein. The processor 202 may also display objects, applications, data, etc. on an interface/display 206. The processor 202 may also receive inputs through the interface/display 206. The processor 202 is also coupled to a transceiver 208. With this configuration, the processor 202, and subsequently the computing device 201, can communicate with other devices, such as the server 225 through a connection 270 and the image capture device 285 through a connection 280. For example, the computing device 201 may send to the server 225 information such as depth information or physiological information of the patient, determined about the occupant by depth data processing.

The computing device 201 may correspond to the computing device of FIG. 1 (computing device 118) and the image capture device 285 may correspond to the image capture device of FIG. 1 (camera 102). Accordingly, the computing device 201 may be located remotely from the image capture device 285, or it may be local and close to the image capture device 285.

In various embodiments disclosed herein, the processor 202 of the computing device 201 may perform the steps described herein. In other embodiments, the steps may be performed on a processor 226 of the server 225. In some embodiments, the various steps and methods disclosed herein may be performed by both of the processors 202 and 226. In some embodiments, certain steps may be performed by the processor 202 while others are performed by the processor 226. In some embodiments, information determined by the processor 202 may be sent to the server 225 for storage and/or further processing.

In some embodiments, the image capture device 285 is or forms part of a remote depth sensing device or depth determining device. The image capture device 285 can be described as local because it is relatively close in proximity to a patient so that at least a part of the patient is within the field of view of the image capture device 285. In some embodiments, the image capture device 285 can be adjustable to ensure that the occupant is captured in the field of view. For example, the image capture device 285 may be physically movable, may have a changeable orientation (such as by rotating or panning), and/or may be capable of changing a focus, zoom, or other characteristic to allow the image capture device 285 to adequately capture the occupant for monitoring. In various embodiments, a region of interest may be adjusted after determining the region of interest. For example, after the ROI is determined, a camera may focus on the ROI, zoom in on the ROI, centre the ROI within a field of view by moving the camera, or otherwise may be adjusted to allow for better and/or more accurate tracking/measurement of the movement of a determined ROI.

The server 225 includes a processor 226 that is coupled to a memory 228. The processor 226 can store and recall data and applications in the memory 228. The processor 226 is also coupled to a transceiver 230. With this configuration, the processor 226, and subsequently the server 225, can communicate with other devices, such as the computing device 201 through the connection 270.

The devices shown in the illustrative embodiment may be utilized in various ways. For example, either of the connections 270 or 280 may be varied. Any of the connections 270 or 280 may be a hard-wired connection. A hard-wired connection may involve connecting the devices through a USB (universal serial bus) port, serial port, parallel port, or other type of wired connection that can facilitate the transfer of data and information between a processor of a device and a second processor of a second device. In another embodiment, any of the connections 270 and 280 may be a dock where one device may plug into another device. In other embodiments, any of the connections 270 and 280 may be a wireless connection. These connections may take the form of any sort of wireless connection, including, but not limited to. Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another embodiment, the various devices may connect through an internet (or other network) connection. That is, any of the connections 270 and 280 may represent several different computing devices and network components that allow the various devices to communicate through the internet, either through a hard-wired or wireless connection. Any of the connections 270 and 280 may also be a combination of several modes of connection.

It will be understood that the configuration of the devices in FIG. 2 is merely one physical system on which the disclosed embodiments may be executed. Other configurations of the devices shown may exist to practice the disclosed embodiments. Further, configurations of additional or fewer devices than the ones shown in FIG. 2 may exist to practice the disclosed embodiments. Additionally, the devices shown in FIG. 2 may be combined to allow for fewer devices than shown or separated such that more than the three devices exist in a system. It will be appreciated that many various combinations of computing devices may execute the methods and systems disclosed herein. Examples of such computing devices may include other types of devices and sensors, infrared cameras/detectors, night vision cameras/detectors, other types of cameras, radio frequency transmitters/receivers, smart phones, personal computers, servers, laptop computers, tablets, blackberries, RFID enabled devices, or any combinations of such devices.

FIG. 3A depicts a 3D rendered image of a patient monitoring system 300, in accordance with an embodiment. The system 300 has a support apparatus corresponding to supporting apparatus 108 described with reference to FIG. 1. The support apparatus is mobile and has a moveable base portion 302, a support body 304 and a support arm. The moveable base portion 302 has wheels and is moveable along a floor to position the system 300 adjacent to a bed, for example. The support arm has a first (vertical) extending portion 306 and a second (vertical) extending portion 308. The first extending portion 306 is connected at to an upper end of the support body 304 by a connecting member 310.

At a distal end of the support arm (at the terminal end of the second extending portion 308) there is provided a fitting 312 for a camera. The support arm is shaped to position the camera at its sensing position, at a height above the bed. At this position, the camera is operable to capture depth data about a desired region of the patient. FIG. 3A also shows display 322 supported by support body 304. It will be understood that the other elements of the patient monitoring system (for example, the processor) are not depicted in FIG. 3A, for clarity.

FIG. 3B depicts the system 300 in a clinical environment in a bedside adjacent position. FIG. 3B depicts system 300 of a configuration of the system 100 and bed 324. As can be observed from FIG. 3B, the support apparatus provides the camera at an elevated position above the bed 324 such that, in use, its field of view includes a region of interest of a patient lying in the bed.

A calibration step may or may not be required. In some non-limiting embodiments, a calibration step is performed. In particular, for some patients, such as neonates, a calibration step may be required. For some patients, for example, adult patients, a calibration may or may not be required. In a further embodiment, the depth camera has a fixed focus depth for cases where the patient is not expected to move and the camera remains at a fixed distance from the patient.

FIGS. 3A and 3B depict the system in one example embodiment. In other embodiments, the projector and camera may be secured in elevated positions using alternative support apparatuses. As a non-limiting example, an arm support secured at a first end to a wall behind a bed supports the camera. In a further non-limiting example, a support apparatus secured to part of the bed or a platform attached to the bed (for example, to the back or side of the bed) is used to support the camera. In a further non-limiting example, a support apparatus for supporting the camera and project is secured at a base to the floor. In a further non-limiting example, a support apparatus is secured to a ceiling above a bed to support the camera at the elevated position.

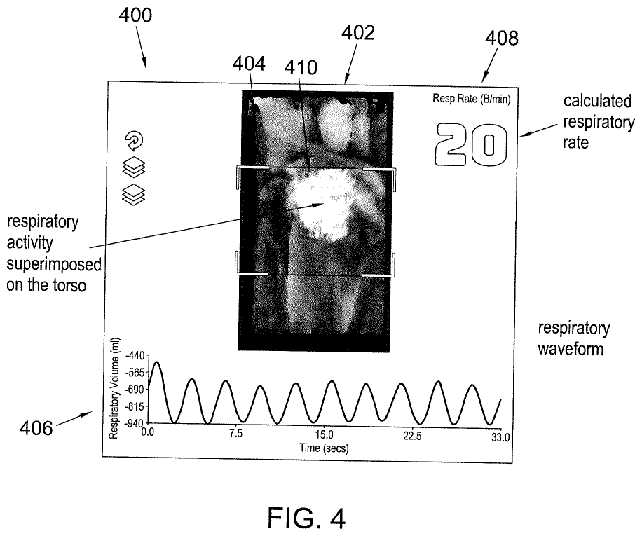

FIG. 4 is a screenshot 400 depicting an image of a screenshot displaying physiological information obtained using depth data. The screenshot 400 includes image 402 captured by a camera according to various embodiments described herein. In particular, image 402 can be considered as an image captured by the depth camera described with reference to FIG. 1 and FIG. 2. The image 402 is a depth image (also referred to as a depth map) captured by the depth sensing camera. The depth image includes depth information about the distance from the camera to any object or subject in the image across the field of view. This type of image or map can be obtained by a stereo camera, a camera cluster, camera array, or a motion sensor. When multiple depth images are taken over time in a video stream, the video information includes the movement of the points within the image, as they move toward and away from the camera over time. The depth data represents depth information across the field of view of the camera, in particular, the depth information is a function of position across the field of view. It will be understood that the depth image depicted is one of a sequence of depth images captured over time as part of a video stream from the camera.

Also depicted in image 402 is a region of interest 404. The image 402 includes a representation of subject (corresponding to subject 104 of FIG. 1) and a region of interest (ROI) 404. In this embodiment, the ROI 404 includes the chest region of the subject. The ROI 404 provides depth data that can allow a respiratory signal or other breathing signal to be determined. The ROI 404 is located about the subject's chest. In this example, the ROI 404 is a rectangular box. In various embodiments, other ROls may be different shapes. Because the image includes depth data, such as from a depth sensing camera, information on the spatial location of the subject, and therefore the subject's chest and the ROI 404, can also be determined.

The depth information can be contained within a matrix, for example, or any suitable mathematical representation. As the patient breathes, their chest moves toward and away from the camera, changing the depth information associated with the images over time. As a result, the location information associated with the ROI 404 changes over time. The position of individual points within the ROI 404 may be integrated across the area of the ROI 404 to provide a change in volume over time.

FIG. 4 also depicts a respiratory waveform 406. The respiratory waveform 406 is determined using the depth information of the depth image. FIG. 4 depicts a graph of volume displacement. The x-axis of graph 404 is time. The y-axis 406 of graph 404 is volume displacement (the sum of depth changes over time). The volume waveform represents the volume of air being inhaled and exhaled from the patient's chest. The volume waveform can be used to extract parameters such as tidal volume, minute volume and respiratory rate. The respiratory signal is a physiological signal obtained by processing depth data, as described with reference to FIGS. 1 and 2.

In FIG. 4 the waveform 406 is a volume waveform. Other waveforms associated with respiratory activity may be obtained from the depth data as part of the respiratory rate algorithm. For example, a respiratory flow waveform may be obtained. As a further example, waveforms that represent changes in volume that correspond to motion towards the camera or changes in volume of motion away from the camera may be used.

In the above-described embodiments, the extraction of physiological information, for example, a physiological signal or other information from the depth data can be performed in accordance with a number of different processing procedures or depth processing algorithms. Examples of depth processing algorithms can be found in “Noncontact Respiratory Monitoring Using Depth Sensing Cameras: A Review of Current Literature”, Sensors, 2/(4), 1135 by Addison, A. P., Addison, P. S., Smit. P., Jacquel, D., & Borg, U. R. (2021). The processor is configured to perform one or more of these depth data processing procedures to obtain physiological information.

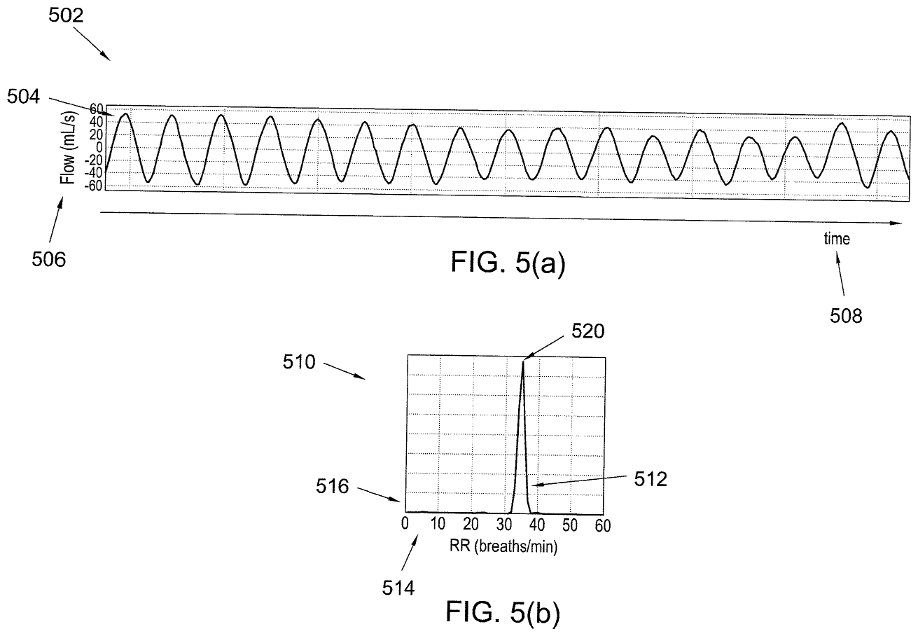

In the present embodiment, as described with reference to FIGS. 5, 6 and 7, the processor is configured to execute one or more depth data processing procedures or algorithms to obtain physiological information. In particular, present embodiments relate to procedures in which at least part of the depth data is processed to derive a time varying signal represented graphically as a waveform. The processor is further configured to perform a power spectrum or frequency analysis on the time varying signal to obtain a power spectrum. The time varying signal and the power spectrum are used to obtain physiological information for the subject.

FIG. 4 shows a graph 406 showing a respiratory waveform determined using the depth information of the depth image, according to various embodiments described herein, in accordance with a depth processing algorithm. To obtain a respiratory rate using the depth camera, the respiratory signal is derived from the depth data. Any respiratory activity in the region of interest manifests itself as a change in distance between the camera to the surface of the patient's torso over time. The changes cycle with the breath period. Frame-to-frame differences in depth are then integrated over the region of interest and used to derive a time-varying signal commensurate or associated with respiratory activity. The result is a respiratory signal that may be represented or described as a waveform. The respiratory signal is found to be robust to noise, posture, bedclothes and other sources of noise.

Graph 406 is a graph of volume displacement. The x-axis of graph 406 is time. The y-axis of graph 406 is volume displacement (the sum of depth changes over time). As can be seen the respiratory waveform has a sinusoidal form. From this waveform a respiratory rate may be calculated. The respiratory rate 408 is determined by the depth processing algorithm. The respiratory rate is calculated, for example, from the waveform or from a further signal derived from the waveform. The algorithm may determine the respiratory rate using respiratory modulations identified in the signal. The algorithm may determine the respiratory rate by performing a power or frequency analysis to determine a power spectrum, as described with reference to FIG. 5. Other physiological parameters may be determined and displayed, for example, indicative minute volume, and indicative tidal volume.

In the above-described embodiments, the system, for example, the system 100 or the system 200, has a display. The display is configured to graphically display depth images representative of the depth information. The display may also display one or more physiological parameters or graphically display the time-varying physiological signal. The display may be further configured to overlay the depth image with colours representative of the determined physiological information. For example, the depth image may be represented over time using a colour change or other change in appearance of the depth image. For example, a first colour may be used for inhalation (for example, overlaying the part of the chest region moving towards the camera) and a second, different colour may be used for exhalation (for example, overlaying the part of the chest region moving away from the camera). Alternatively, a first visual aspect, for example, colour may be used to indicate inhalation only.

In some embodiments, the depth data processing procedure is configured to determine a mask for a visual overlay to visually represent exhalation or inhalation, for display. An example of a graphical representation of a determined mask is depicted in FIG. 4 as visual overlay 410. It will be understood that a number of methods may be used to determine the mask, for example, as described in US Patent Publication No. 2023/082016 A1. In some embodiments, the determination and/or display of the mask may be dependent on the setting of the NRM flag.

In some embodiments, the determined mask may also be used to determine the signal. In some embodiments, a processed or modified version of the determined mask may be used to determine the signal. For example, the processed version may result from an in-filling process or averaging process. However, it will be understood that the signal can also be determined without using a mask, for example the signal may be obtained using depth information obtained and integrated across the whole scene. With reference to FIG. 4, a target boundary is defined, in that example a target box or rectangle, and the depth data within the target boundary is processed to obtain the signal.

In some embodiment, the respiratory rate algorithm includes determining a mask and generating a mask overlay visualisation to visually represent exhalation or inhalation. A mask may be determined in a number of different ways. As an example, a mask may be a spatial coherence mask using the fact that different regions of the depth field behave differently, particularly, that for respiration, the changes in the depth field are coherent (e.g., they tend to move in the same direction, towards or away from the camera, and for background or a stationary surface, the changes in the depth field are incoherent (e.g., they tend to have a “zero” center). Such a Spatial Coherence mask may be calculated by calculating a recent change in depth for each of multiple regions of the depth field and assigning one of two binary values based on the direction of depth change, ignoring the magnitude of the change. A filter (e.g., box filter) is applied to the values and the local bias in the depth field is calculated effectively as a measure of how coherent the changes are in the depth field surrounding each point in the depth field. If the noise is incoherent, the local bias is small (e.g., close to 0), compared to a region where the torso is clearly moving towards or away from the camera(s), the local bias is large (e.g., close to 1). Small values (e.g., with a cut off at e.g., less than 0.2 or less than 0.4) can be removed from the local bias and the resulting trimmed local bias mask can be used to highlight respiration in the depth field as the spatial coherence mask. In general, the determination of the mask may include assigning regions having coherent changes to a mask calculation and withholding regions having incoherent changes from the mask calculation.

After determining the mask, it is known which regions of the depth field correspond to respiration. The mask is used to extract the depth data from only the regions corresponding to respiration; from this, a visual overlay, which can be color coded to represent moving toward the camera (inhale) and moving away from the camera (exhale). In some embodiments, the mask can be used to generate a visualisation overlay. For example, a green overlay can be shown, whereas when the ROI region is moving away from the camera (e.g., on an exhale), no color overlay is shown. In other implementations, the user or viewer of the monitoring system can select the settings of the visual output. For example, the user may desire a green overlay for an inhale and a red overlay for an exhale, or, a white overlay for an inhale and no color overlay for an exhale, e.g., for users that are red/green colorblind. In some arrangements, the strength, tone, or brightness of the selected color may change as the movement (e.g., distance) changes. For example, the user may desire a green overlay for an inhale and a red overlay for an exhale, or, a white overlay for an inhale and no color overlay for an exhale, e.g., for user that are red/green colorblind. In some arrangements, the strength, tone, or brightness of the selected color may change as the movement (e.g., distance) changes.

As described above, an algorithm for determining physiological information may use both a time varying signal derived from depth data and a power spectrum, or other transformed signal, obtained from the time varying signal. FIG. 5(a) depicts a first plot 502 depicting an example flow waveform 504 obtained using a respiratory rate algorithm. Plot 502 has an x-axis of time 506 and a y-axis of flow (units of mL per second) 508. The flow waveform is obtained during a period of respiratory movement, in particular, a period of constant respiratory rate of 35 breaths per minute. As can be seen from FIG. 5(a), the waveform represents a time-varying signal having sinusoidal characteristics.

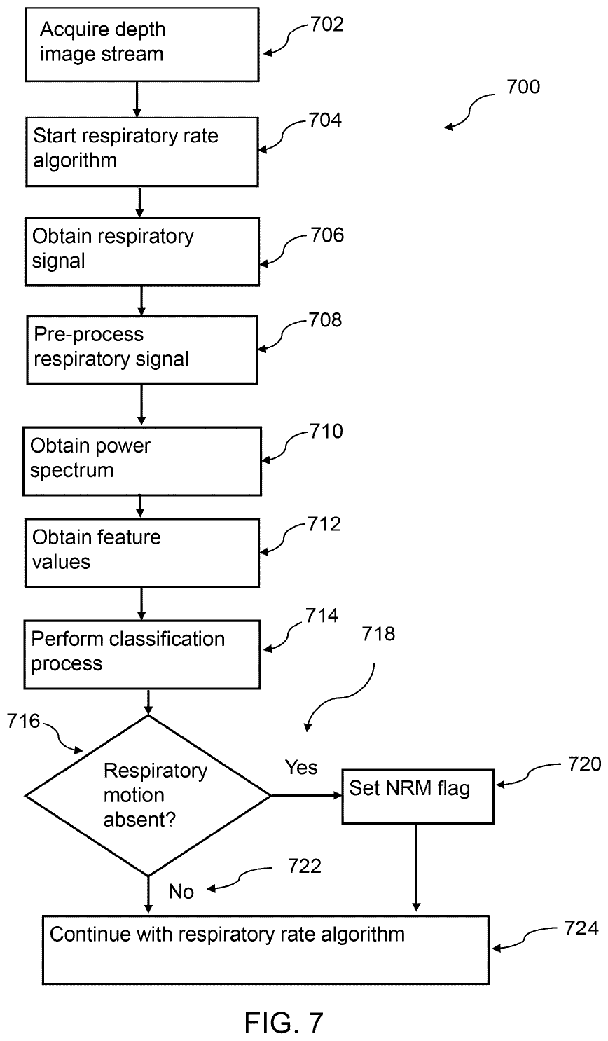

FIG. 5(b) depicts a second plot 510 depicting a power spectrum 512 obtained from the waveform 504 of FIG. 5(b). The power spectrum 512 is plotted against an x-axis of respiratory rate (units are breaths per minute) 514 and a y-axis. The power-spectrum may be as defined with reference to step 710 of FIG. 7. As can be observed from FIG. 5(b) the constant respiratory rate of 35 breaths per minute is represented by a power spectrum having a single peak 518 centred at a respiratory rate of 35 breaths per minute.

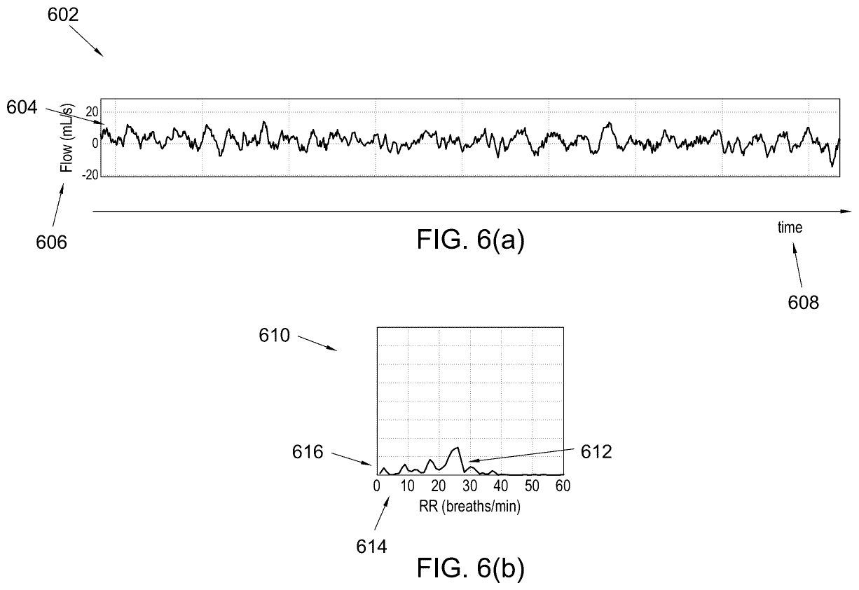

In contrast to FIG. 5(a) and FIG. 5(b), FIG. 6(a) and FIG. 6(b) depict a waveform and power spectrum during a period where there is no respiratory movement. FIG. 6(a) depicts a first plot 602 depicting an example flow waveform 604 obtained using a respiratory rate algorithm. Plot 602 has an x-axis of time 606 and a y-axis of flow (units of mL per second) 608. The flow waveform is obtained during a period of no respiratory movement. As can be seen from FIG. 6(a) the waveform represents a time-varying signal.

FIG. 6(b) depicts a second plot 614 depicting a power spectrum 612 obtained from the waveform 604 of FIG. 6(a). The power spectrum 612 is plotted against an x-axis of respiratory rate (units are breaths per minute) 614 and a y-axis. In contrast to the power spectrum 512 of FIG. 5(b), the power spectrum 612 has low power and does not have a single well-defined peak.

FIG. 7 is a flowchart of a method 700 of setting a no-respiratory motion (NRM) flag for a depth data processing procedure, in the present embodiment for a respiratory rate algorithm. While FIG. 7 depicts a method of setting a flag for a respiratory rate algorithm it will be understood that the method may be applied to other physiological and/or contextual information obtaining algorithms. The method is performed by the system described with reference to FIGS. 1 and 2, however, it will be understood that other system configurations may be used. In this embodiment, a value of “true” for the flag means that no respiratory motion is detected in the scene or field of view. This corresponds to cases when respiration has stopped or the subject has left the scene.

At step 702, images are captured by the depth sensing camera 110 in the form of streamed video feeds. These video feeds contain depth data, as described with reference to FIGS. 1 to 4.

At step 704, a respiratory rate algorithm is performed. The respiratory rate algorithm determines respiratory rate using the obtained depth data. To determine the respiratory rate, the processor processes the depth data to derive a time varying signal at step 706. The respiratory signal is obtained substantially as described with reference to FIG. 4.

At step 708, one or more pre-processing steps are performed on the respiratory signal. In other embodiments, no pre-processing steps are performed. The pre-processing steps can include a number of steps that enhance properties, features or characteristics of the respiratory signal for the power spectrum calculation to enhance characteristics that help in the classification of the signal. This pre-processing may consist of the application of one of several filters (including averaging, bandpass filter, high-pass filter, low-pass filter, Kalman filter). The pre-processing stage may also include the removal of outlier samples by setting them to NaN (invalid) through different techniques (for example, values above/below a percentile value, values above maximum power or below minimum power, values above an absolute threshold or below an absolute threshold. In some embodiments, part of the signal is removed, for example, a removal of additional samples adjacent to invalidate the signal. In some embodiments, a portion of signal is removed from each side of the signal. The pre-processing comprises filtering/selecting and/or cleaning such as these. The samples refer to the individual points that constitute the flow/volume signals. It may happen, for example, due to motion or some gross artefact that the flow waveform contains such outliers. Setting those samples in the flow/volume waveform as NaN may avoid an incorrect RR calculation.

At step 710, a power spectrum is obtained. The power spectrum may also be referred to as a power spectral density (PSD). In the present embodiment, a power spectrum analysis is performed on the respiratory signal. In some embodiments, other frequency dependent signals are obtained from the respiratory signal. Alternative transformations to a frequency domain can be used. The further signal derived from the time dependent signal can be a function of frequency and/or a power or energy per unit time signal.

A number of known signal processing methods to obtain the power spectrum from the respiratory signal may be used. In an example, a Fast Fourier Transform (FFT) of the respiratory signal is obtained and the square of the absolute value or magnitude of the FFT is obtained to provide a power spectral density. The power spectral density represents how the power of the signal is distributed over frequency. The power spectral density represents a distributed of power of the signal over frequency. The power spectral density can be integrated between two frequencies to obtain the power in the signal over that range. Further examples on calculating the power spectrum or power spectral density may be found at, for example, at https://uk.mathworks.com/help/signal/ug/power-spectral-density-estimates-using-fft.html.

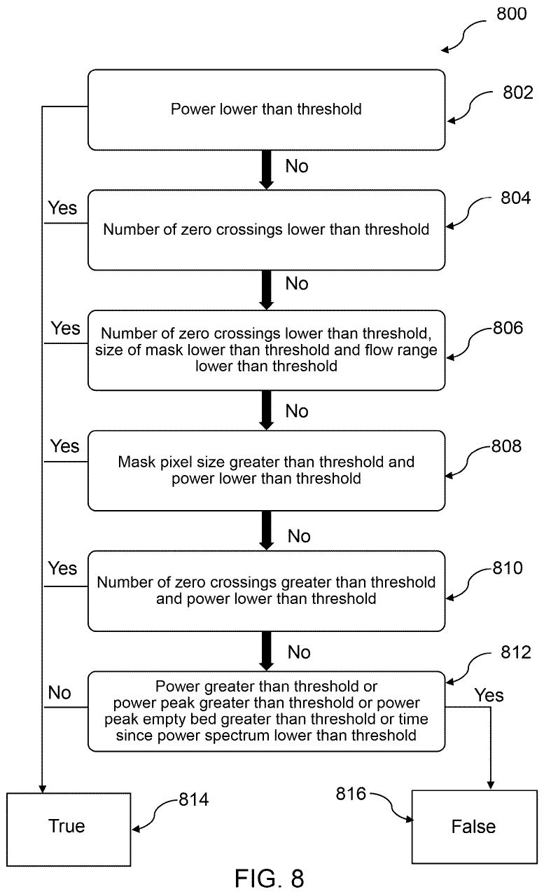

At step 712, further information from the depth data and the waveform and the power spectrum is obtained. In the embodiment of FIG. 7, three feature sets are used for classifying no respiratory motion in the scene (depth data features, power spectrum features and waveform features). The further information thus includes values for a number of pre-determined features for the waveform, the power spectrum and the depth data. It will be understood that different combinations of features can be used for the classification method. An example of a classification method and the corresponding features values are described with reference to FIG. 8. In some embodiments, the features used may be derived from the respiratory signal and/or the corresponding power spectrum or from the depth data.

The following non-limiting list of features that provide further information can be used in a classification process. In general, signals derived from the depth data, for example, the time varying respiratory signal and the power spectrum signal can provide a source for further information for the classifier. Features relating to an area under at least part of, optionally all, of the signal can be used. Features relating to an average value for at least part of, optionally all of the signal can be used.

Features relating to one or more properties associated with or derived from one or more peaks and/or troughs of the signal can be used. For example, such properties include an area of one or more peaks; a height of one or more peaks; a spread of one or more peaks; a number and/or density of peaks in the signal; a ratio between a property of one peak and the same property of another peak. In such examples, the area may be an integral or summed quantity of at least part of the signal. The height may be an intensity of magnitude. The spread may correspond to a time or frequency range. The number and/or density of peaks may be determined relative to a threshold value. The at least one property may comprise a relative area, size or spread of a first peak to a second peak. Other features relating to a property associated with a zero crossing or other threshold crossing of the signal can be used. These properties include, for example, a number or density of zero crossings. Features relating to a measure of bias or noise in the signal can be used. Such a feature may be obtained by processing the signal to obtain a measure of bias or noise.

In general, features relating to a count, location or density of one or more identified features in the signal can be used or features relating to any other further mathematical property of the signal can also be used.

Regarding the time varying signal or waveform, features associated with one or more respiratory signals derived from the depth data can be used. The respiratory signals include signals such as a respiratory flow or volume waveform.

In the embodiment of FIG. 7, these features are calculated from a time window applied to the waveform. The time window can be, for example, 30 s to 60 s. In some embodiments, the time window is in the region up to 120 seconds. As described above, the waveform may be pre-processed before the feature extraction process. Non-limiting examples of the waveform features include: features representative of a number and/or density of zero crossings; a measure of the amplitude or size of the waveform; a measure of bias and/or noise in the signal; a property of one or more peaks and/or troughs of the waveform. The density of zero-crossings can be determined by dividing the number of zero-crossings by the duration of the time window of analysis. The amplitude can be determine using a number of methods, for example, using an interpercentile range. The number of peaks and/or troughs may be determined relative (i.e. above or below) a pre-determined threshold value. A measure of bias can be useful for particular signals, for example, a respiratory volume signal displays a trend over time when no breathing is present due to noise being biased.

Turning to the power spectrum, further information from the power spectrum can be used as inputs for a classifier model. The following non-limiting examples of power spectrum features are provided. As a first example, the total power in the spectrum. This can be determined, for example, by integrating or otherwise summing an area under the power spectrum. Properties of one or more peaks can also be used. As an example, a total power contained within a main peak and/or a second peak in the power spectrum can be used. Such a total power can be calculated by identifying the frequency range of each peak and integrating or otherwise summing the power in the frequency range. As a further example, a number of peaks in the power spectrum above a pre-determined threshold can be used. As a further example, a percentage of power in a main peak or a second peak can be used. For example, the total power contained in a peak divided by the total power of the power spectrum. As a further example, a relative power of a peak in the spectrum can be used. This may be calculated by calculating a ratio power between a first identified peak and a second identified peak in the power spectrum. In a further example, a location of the peak, in this embodiment, can be used. For example, the frequency of the main peak in the spectrum (the dominant frequency) can be used and/or the frequency of any other significant peaks.

The window length for a waveform may be the same as the window for the power spectrum features. Alternatively, the window length for the waveform may be different to a window length for the power spectrum. As an example, a longer window may be used to obtain properties of peaks in the waveform, while a shorter window may be used to obtain properties of peaks in a power spectrum. As a further example, a window length of 120 seconds may be used for the waveform and a window length of 30 seconds may be used for the power spectrum.

In some embodiment, values for the same features for several different window lengths may be extracted to identify the behaviour of the signal at different time scales. As an example, one or more of the features listed above may be obtained for a window of a first duration (for example, 15 seconds) and for a window of a second duration (for example, 60 seconds) in order to generate twice as many features to be used in the classifier. Since the features depend on the respiratory rate (if present), it may be advisable to use a longer window when a subject is present and breathing slowly. However, if a patient is breathing faster, a shorter window may be more efficient. Since the RR of the subject is not known a priori, using windows of different sizes allows information from more features to be combined. This may offer improved accuracy for better accuracy in determining the absence of breathing.

In addition to the signal and power spectrum features listed above, further information can be extracted from the depth data in the field of view (i.e. the depth matrix acquired by the depth camera) may be used. These features of the depth data may be used in addition or as alternative to the signal and power spectrum features. The following non-limiting examples of depth data feature are provided. As a first example, a measure of an average or a summed value of depth over at least part of the field of view can be calculated or used. Other features derived directly from the depth data can be used as an input to the classifier. As a non-limiting example, depth features may be combined into new features. As a non-limiting example, a feature can be constructed that is the average value of the depth (for example, the average value of the depth across the region of interest) multiplied by the square of the pixel size.

The procedure comprises processing the depth data to define a visualisation mask representing a moving area of movement away and/or towards the depth sensing camera in the field of view, and wherein a property of the identified area is used to determine the absence of respiratory motion. The property comprises a size and/or fill ratio and/or a measure of movement toward the depth sensing camera and/or a measure of movement away from the depth sensing camera and/or a measure of confidence intervals and/or a mask fill ratio.

In some embodiments, further information including one or more properties of a determined mask is used to determine the absence of respiratory motion in the field of view. These properties can be provided as input features to the classifier. As non-limiting examples, the properties include the size of mask, for example, a pixel area of the calculated mask. A smaller mask may indicate a smaller probability of respiration being present. In addition, the properties may include a mask fill ratio, for example, a measure of how much of the mask has valid data; a mask G fill ratio corresponding to a measure of the mask has “positive” movement—i.e., how much of the mask is “green” in the UI and how much is moving towards the camera; a mask R fill ratio corresponding to how much of the mask has “negative movement—i.e., how much of the mask is “red” in the UI—how much is moving away from the camera. Other mask parameters can also be used, for example, a mask mean or other average can be used. The determined mask is not a binary mask: it has a range of values and may be considered as a confidence metric. Therefore, the mean of the mask values can be thought of as an overall confidence metric.

With regard to using a measure of valid data, it will be understood that the depth data that is obtained by the camera is not 100% filled such that some of the pixels do not provide any depth information. This may occur for various reasons, for example, the failure of algorithm inside the camera, or due to poor contrast in the IR images used for the disparity calculation. As a further example, “shadows” around objects may be observed, when only one of the cameras can see a spot in the image and so the depth is unresolved. Such artefacts may be detected and their values are marked invalid. A measure of valid data can be obtained by counting the number of pixels that have returned valid data. This measure may be expressed as a percentage. The count may be further processed over time to smooth the measure.

Other mask parameters and metrics can be used, for example, properties of the size of the mask, the values inside the mask, or the shape of the mask can also be used. For example, a measure of circularity of the mask could be used. A very long, thin mask is unlikely to represent true respiration and therefore may provide an input feature for the classifier.

At step 714, a classification process is performed using the features obtained from step 712. The classification process determines the absence of respiratory motion in the field of view. The classification process is described in further detail with reference to FIG. 8.

At step 716, an absence of respiratory motion is determined. If the respiratory motion is absent (step 718) the method proceeds to set a flag, referred to as the no respiratory motion flag at step 720. The method then proceeds to continue with the respiratory algorithm at step 724. If respiratory motion is present (step 722) then the respiratory algorithm continues at step 724 without setting a flag.

In the embodiment of FIG. 7, the operation of the respiratory algorithm is dependent on the value of the flag. In particular, the respiratory algorithm displays a respiratory rate depending on the value of the flag. The flag may be used by the respiratory algorithm or may be stored and used by a further algorithm. For example, in some embodiments, a separate procedure for displaying physiological information may be executed and run independently, for example, on a separate processor, from the procedure that generates the time varying signal and/or further signal and performs the depth data processing. In some embodiments, additional physiological information is displayed depending on the flag.

In some embodiments, the determination of the absence is repeated over a pre-determined time period and the results are stored in a memory storage buffer, for example, in memory 204. In such an embodiment, the classification can be performed at a first rate (as a non-limiting example, every second) and the results are saved to the memory storage buffer. The determination of no respiratory motion and the setting of the flag is then dependent on a certain proportion of the stored classifications (for example, 80%) being indicative of no respiratory motion. The buffer can be any suitable time period in length including 10 seconds, 20 seconds, 30 seconds. The buffer allows smoothing of miscalculations and minimisation of flicker.

FIG. 8(a) is a flowchart of a classification procedure 800 in accordance with an embodiment. Within the workflow of FIG. 7, the classification procedure is performed at step 714. The classification procedure is based on pre-determined thresholds for a number of features of the waveform and power spectrum and depth date. A sequence of evaluation steps are performed using feature values (labelled 802, 804, 806, 808, 810, 812). In the present embodiment, the evaluation steps correspond to a set of conditions, in particular threshold conditions. If any of the conditions are satisfied the classification procedure results in a positive classification at step 814. The positive classification corresponds to an absence of respiratory motion. If a condition in the sequence is not satisfied the method proceed to test the next condition. If none of the conditions are satisfied, the classification procedure results in a negative classification at step 816 (corresponding to a presence or a lack of absence of respiratory motion). The classification process described in the following is an example of a whitebox classification process in which all variables and thresholds are well defined and explicit.

The threshold can be determined using different methods. As a first example, by evaluating the performance of each feature versus the expected behaviour (i.e. by performing a grid search of each feature). As a second example, a binary tree may be used to optimizing using labelled regions with motion/no-motion. As a third example, a neural network may be used and trained to obtain threshold values.

Prior to step 714, the required further information is extracted from the waveform and power spectrum. In this example, the following further information is obtained and provided as input to the classification procedure. From the waveform: the number of zero crossings of the waveform, the interpercentile range of the flow in the window (e.g. 5-95th percentile). The interpercentile range of the flow in the window provides a robust measure of the amplitude of the flow. From the power spectrum, integral of the power spectrum in frequencies from 0 to 60 brpm, integral around the biggest peak in the power spectrum (+/−2 samples from the maximum). From the depth data/determined mask: total size of the respiratory mask from which the flow waveform is calculated. This may be the respiratory mask that is displayed as a visual overlay or a processed or filtered version of that mask. In addition to the depth data, power spectrum and waveform features additional further information obtained during the depth data processing procedure may be used. In the present embodiment, a time in seconds since there was a post of respiratory using the power spectrum is used an input to the classifier.

It will be noted that the same feature can be used more than once. The threshold can be dependent on the value of other features (for example, use one threshold for the feature if a power is below a certain value and use a different threshold if the power is above a certain value).

TABLE 1

| | | | Description of Parameter |
|---|---|---|---|
| Name | Code Variable Name | Value | relating to threshold |
| T01 | threshold zero_ | 80 | the number of times the flow |
| | crossings_0_1 | | signal crosses the line of flow = 0 |
| T02 | power total_ | 2 | integral of the power spectrum in |
| | emptybed_ | | frequencies from 0 to 60 brpm |
| | threshold_0_2 | | |
| T11 | Threshold_zero_ | 70.5 | the number of times the flow |
| | crossings_1_1 | | signal crosses the line cf flow = 0 |
| T21 | mask_pixel_size | 0.035 | total size of the respiratory mask |
| | threshold_2_1 | | from which the flow waveform is |
| | | | calculated |
| T22 | flow_range_robust | 24.6 | interpercentile range of the flow |
| | threshold_2_2 | | in the window (e.g. 5-95th |
| | | | percentile) |
| T31 | flow_range_robust_ | 28.6 | interpercentile range of the flow |
| | threshold_3_1 | | in the window (e.g. 5-95th |
| | | | percentile) |
| T32 | Power_total_empty_ | 4.4 | integral of the power spectrum in |
| | bed_threshold_3_2 | | frequencies from 0 to 60 brpm |
| T33 | power_peak_ | 0.5 | integral around the biggest peak |
| | emptybed_ | | in the power spectrum (+/−2 |
| | threshold_3_3 | | samples from the maximum) |
| T41 | threshold_power_ | 500 | integral of the power spectrum in |
| | total_emptybed | | frequencies from 0 to 60 brpm |
| T42 | threshold_power_ | 0.75 | integral of the power spectrum in |
| | total_emptybed | | frequencies from 0 to 60 brpm |
| T43 | threshold_power_ | 0.5 | integral around the biggest peak |
| | peak_emptybed | | in the power spectrum (+/−2 |
| | | | samples from the maximum) |
| T44 | Min_time_since_ | 34 | time in seconds since there was |
| | last_rr_ps | | a post of RR using power |
| | | | spectrum |

In Table 1 there are references to a sample, in particular to plus or minus a number of samples from a maximum. It will be understood that in this context a sample in the power spectrum has a resolution of 1 breath per minute.

In Table 1, “Min_time_since_last_rr_ps” refers to a minimum time since last RR post. If the power spectrum alone is confident enough, the PS information may be used alone to post a RR value (even if there are no valid breaths). If there is no viable power spectrum information for more than X seconds, then this can be used as a feature.

Turning to FIG. 8, condition 802 corresponds to evaluating if the power is lower than T02. Condition 804 corresponds to evaluating if the number of zero crossing is lower than T01.

Condition 806 corresponds to a combined condition (an AND operation). Condition 806 corresponds to evaluating if the number of zero crossings is lower or equal to T11 and the size of the mask pixel is lower than T12 and the flow range is lower than T31.

Condition 808 corresponds to evaluating if the mask pixel size is greater than T21 and the power is lower than T32.

Condition 810 corresponds to evaluating if the number of zero crossings is greater than T11 and the flow range is lower than T22 and the power is lower than T33.

Condition 812 corresponds to evaluating if the power is greater than T41 or the power peak (power_peak_RR) is greater than T42 or the power peak empty bed is greater than T43 or the time since PS is lower than T44.

The classification method of FIG. 8 provides an example of a classification process thresholds and features that may be used. In a method, for the classification of no respiratory motion, a power spectrum window may be calculated from a respiratory signal derived from the depth camera system. This can be, for example, a respiratory flow signal or its integral, the volume signal. The power spectrum must have characteristics tuned to the evaluation of whether there is (or is not) respiratory motion in the window of analysis. An example of this could be specifying an adequate window duration (e.g., 30 seconds, or 60 seconds or any power spectrum that is sufficiently long to include enough breaths). The window size can be selected to provide a signal of sufficient strength. If the window is too small, the power spectrum may not find a frequency and detect a band with a single peak. On the other hand, if the window is too long, because RR may not be at a constant frequency, the window may include more frequencies and make the power spectrum less sharp around a single frequency.

In the above-described embodiments, a classification method is described. It will be understood that a machine learning classification method can be used. Such a system may be configured similar or identical to the system 100 shown in FIG. 1, but with the addition of a machine learning module in the computing device (for example, as part of the processor or memory 126).

The network used for machine learning based may be developed through transfer learning of existing neural networks, such as Alexnet, ResNet, GoogLeNet, etc., or it may be developed from scratch and include layers such as input layer, convolutional layer, relu layer, pooling layers, dropout layers, output layers, softmax layer, etc. The machine learning model can be trained based on a data set of patient images where at least some of the patient images are pre-labeled with an accurate/correct patient position. The pre-labeled images allow for the machine-learning model to be trained to identify non-labeled images. In some embodiments, the machine-learning model can be trained to identify one or more of the following positions: left, right, prone, supine, sitting, no patient, and multiple persons.

The machine learning model may be any suitable machine learning derived technique. For example, the classification process may include a machine learning derived model and/or classifier, for example, decision tree, kNN (k-Nearest Neighbours), AdaBoost, Random Forest, Neural Network, SVM (support vector machine).

In the above described embodiments, image capture devices and associated depth sensors are described. In an embodiment, the image capture device detects light across the visible spectrum. In an embodiment, the camera detects light in only a portion of the visible spectrum, and/or in the infrared spectrum as well.

The systems and methods described herein may be provided in the form of tangible and non-transitory machine-readable medium or media (such as a hard disk drive, hardware memory, etc.) having instructions recorded thereon for execution by a processor or computer. The set of instructions may include various commands that instruct the computer or processor to perform specific operations, such as the methods and processes of the various embodiments described herein. The set of instructions may be in the form of a software program or application. The computer storage media may include volatile and non-volatile media, and removable and non-removable media, for storage of information such as computer-readable instructions, data structures, program modules or other data. The computer storage media may include, but are not limited to, RAM, ROM, EPROM, EEPROM, flash memory or other solid-state memory technology, CD-ROM, DVD, or other optical storage, magnetic disk storage, or any other hardware medium which may be used to store desired information and that may be accessed by components of the system. Components of the system may communicate with each other via wired or wireless communication. The components may be separate from each other, or various combinations of components may be integrated together into a medical monitor or processor, or contained within a workstation with standard computer hardware (for example, processors, circuitry, logic circuits, memory, and the like). The system may include processing devices such as microprocessors, microcontrollers, integrated circuits, control units, storage media, and other hardware.

The above embodiments relate to depth data processing procedures and algorithms for obtaining physiological signals. While physiological signals relating to respiration are described above, other physiological signals can be determined by the procedure. For example, a pulse rate, oxygen saturation or other vital sign signal. Posture information and sleep apnea information can also be determined. Contextual information such as presence information or activity information. Subject identity information may also be obtained by the depth data processing procedure, in some embodiments.

In the above-described embodiments, the method may comprise performing at least one action in dependence on said flag. The at least one action may comprise triggering an alarm signal. The alarm signal may be a visual or audible alarm. Such a flag informs one or more steps in a RR, other algorithm, so that a respiratory rate is not posted when the flag is on. Such a flag indicates that the signal is not sufficient to be confident that any motion is from respiration. The signal may also indicate more than just a poor quality respiratory signal. For example, in theory, an alarm may be triggered based on the no respiratory motion flag, for example, to inform that a bed may be empty and/or a subject may have stopped breathing.

A skilled person will appreciate that variations of the enclosed arrangement are possible without departing from the invention. Accordingly, the above description of the specific embodiment is made by way of example only and not for the purposes of limitations. It will be clear to the skilled person that minor modifications may be made without significant changes to the operation described.

What is claimed is:

1. A computer-implemented method of performing depth data processing to obtain physiological information from depth data, comprising:
obtaining the depth data, wherein the depth data represents a depth across a field of view;
deriving at least one signal from the depth data;
obtaining further information from at least one of: the depth data or the at least one signal derived from the depth data;
based on the further information, determining an absence of respiratory motion in the field of view; and
setting a flag based on the determining of the absence of respiratory motion in the field of view.

2. The method of claim 1, wherein the depth data processing is configured to extract a signal related to respiration from the depth data.

3. The method of claim 1, wherein the at least one signal derived from the depth data comprises a time dependent signal and a frequency dependent signal, and wherein the further information comprises one or more of: information obtained from the time dependent signal, information obtained from the frequency dependent signal, or information obtained from the depth data.

4. The method of claim 1, wherein deriving the at least one signal comprises deriving a time dependent signal from the depth data, wherein the at least one signal is represented by a waveform, and wherein the further information comprises one or more properties of the waveform.

5. The method of claim 4, wherein the at least one signal comprises a further signal obtained from one of: a power spectral analysis or a frequency analysis of the time dependent signal, and wherein the further information comprises one or more properties of the further signal.

6. The method of claim 1, wherein the at least one signal comprises a respiratory signal and a power spectrum signal obtained from the respiratory signal, and wherein the further information comprises one or more properties of at least one of: the depth data, the respiratory signal, or the power spectrum signal.

7. The method of claim 1, wherein the further information comprises information derived from at least one of: a first signal obtained from the depth data over a first time window or a second signal obtained from the depth data over a second time window.

8. The method of claim 1, wherein the physiological information comprises a physiological signal including at least one of: a respiratory rate, a pulse rate, a tidal volume, a minute volume, an oxygen saturation, a breathing parameter, a breathing effort, posture information, or sleep apnea information.

9. The method of claim 1, wherein the further information represents at least one of a property, a characteristic, or a feature of at least one of the depth data or the at least one signal.

10. The method of claim 1, wherein the further information for the at least one signal comprises at least one of:
a) an area under at least part of the signal;
b) an average value for at least part of the signal;
c) one or more properties associated with or derived from at least one of peaks of the signal or troughs of the signal;
d) a property associated with a zero crossing of the signal;
e) a measure of at least one of bias in the signal or noise in the signal;
f) a count of one or more features in the signal or a density of one or more features in the signal; or
g) a mathematical property of the signal.

11. The method of claim 1, wherein the depth data processing further comprises at least one of:
determining one or more portions of the depth data corresponding to coherent changes or obtaining a mask for a visual overlay;
wherein the further information comprises at least one property of the determined one or more portions or the obtained mask, and wherein the at least one property comprises at least one of: a size, a shape, or a fill ratio.

12. The method of claim 1, wherein the determining of the absence of respiratory motion in the field of view comprises classifying the further information as representative of the absence of respiratory motion in the field of view.

13. The method of claim 12, wherein the classifying comprises using one or more of a machine learning derived model, a classifier, a decision tree, a k-Nearest Neighbors (kNN) algorithm, an Adaptive Boosting (AdaBoost) algorithm, a Random Forest, a Neural Network, or a Support Vector Machine (SVM).

14. The method of claim 1, wherein the determining of the absence of respiratory motion in the field of view comprises applying a threshold based algorithm to compare the further information to one or more thresholds, and wherein determining the one or more thresholds for the threshold based algorithm comprises using training data.

15. The method of claim 1, wherein the depth data processing further comprises displaying the physiological information, and wherein the displaying of the physiological information is responsive to the setting the flag.

16. The method of claim 1, wherein the further information relates to one or more signals derived from the depth data, and wherein the method further comprises:
pre-processing the one or more signals; and
obtaining the further information from the pre-processed one or more signals, wherein the pre-processing comprises at least one of filtering, selecting, or cleaning of the one or more signals.

17. The method of claim 1, wherein the method further comprises:
repeating the determining of the absence of respiratory motion in the field of view; and
storing results of the repeated determining in a memory storage buffer, wherein the setting of the flag is based on an evaluation of stored results in the memory storage buffer.

18. An apparatus comprising:
a depth sensing device configured to obtain depth data representing depth across a field of view; and
a processing resource configured to:
perform depth data processing to obtain physiological information from the depth data, wherein the depth data processing comprises deriving at least one signal from the depth data;
obtain further information from at least one of: the depth data or the at least one signal derived from the depth data;
based on the further information, determine an absence of respiratory motion in the field of view; and
based on the determining the absence of respiratory motion in the field of view, set a flag.

19. The apparatus of claim 18, wherein the depth sensing device comprises at least one of:
a depth sensing camera, a stereo camera, a camera cluster, a camera array, or a motion sensor.

20. A non-transitory machine-readable medium having instructions recorded thereon for execution by a processor to perform a set of operations, comprising:
performing depth data processing to obtain physiological information from depth data, wherein the depth data represents depth across a field of view;
deriving at least one signal from the depth data;
obtaining further information from at least one of: the depth data or the at least one signal derived from the depth data;
based on the further information, determining an absence of respiratory motion in the field of view; and
setting a flag based on the determining the absence of respiratory motion in the field of view.
